(19) United States
(12) Patent Application Publication
(10) Pub. No.: US 2023/0119416 A1
(43) Pub. Date: Apr. 20, 2023

(54) SELF-COMPENSATED FUNCTIONAL PHOTOACOUSTIC MICROSCOPY

(71) Applicant: CITY UNIVERSITY OF HONG KONG, Kowloon (HK)

(72) Inventors: Lidai WANG, Kowloon (HK); Jingyi ZHU, Kowloon (HK)

(21) Appl. No.: 17/504,483

(22) Filed: Oct. 18, 2021

(51) Int. Cl. A61B 5/00; A61B 5/145; G16H 30/20

(52) U.S. Cl. CPC A61B 5/0095; A61B 5/14542; A61B 5/7278; A61B 5/7225; G16H 30/20; A61B 2576/00

(57) ABSTRACT

A method of adjusting for the accuracy of a photoacoustic microscope image of blood oxygen saturation, comprising the use of two wavelengths to monitor the blood oxygen saturation level, and a further reference wavelength to provide an indication of wavelength-dependent fluence loss, and adjusting the blood oxygen saturation by linearization from the fluence loss.

### FIELD OF INVENTION

The invention relates to photoacoustic microscopy systems.

### BACKGROUND OF THE INVENTION

Blood oxygen saturation (sO₂) is of great significance for normal tissue function and disease progression.

The most common approach to measuring blood oxygen saturation is that used in transmissive pulse oximetry. In this approach, a device passes two wavelengths of light through a body part to a photodetector. One of the wavelengths is usually 660 nm, which is more highly absorbed by oxyhemoglobin than deoxyhemoglobin, and the other wavelength is 940 nm, which is more highly absorbed by deoxyhemoglobin than oxyhemoglobin. The amount of light of each wavelength absorbed during the passage is indicative of the respective blood component. The fraction of oxyhemoglobin over the combined amount of oxyhemoglobin and deoxyhemoglobin provides the oxygen saturation. In some situations, it is important to obtain visual mapping of oxygen level in the blood vessels, such as in studies of neural activities via neurovascular coupling, monitoring the recovery of ischemia stroke patients, tracking diagnostic biomarkers for cancer, and monitoring sO₂ during surgery for deprivation of oxygen in specific parts of major organs such as the brain and the heart. Unfortunately, pulse oximetry provides does not provide two-dimensional images or three-dimensional images of blood oxygen levels in tissues.

Various imaging technologies have been used to map sO₂ in three dimensions, such as near-infrared spectroscopy (NIRS) and diffuse optical tomography (DOT) which uses diffusely reflected light at multiple wavelengths to calculate blood oxygen saturation in different points inside tissues to reconstruct a three-dimensional image of blood distribution. However, the spatial resolution of these imaging techniques is poor, as the incident beam of light required to penetrate into biological tissues is scattered more and more as it penetrates into deeper regions of the tissue, which produces poor images of such deeper regions. On the other hand, functional magnetic resonance imaging (fMRI) and positron emission tomography (PET) have been used to measure tissue oxygenation in clinics. However, their sensitivities are limited, and their resolutions are insufficient to resolve images of tiny microvasculature.

Tomography is a method of generating three-dimensional images of biological tissues. The technique involves the obtaining images of sequential two-dimensional sections across a tissue, known as A-lines, through the use of any kind of penetrating wave, and assembling the A-lines into a three-dimensional image. In many cases, the production of these images is based on a mathematical procedure. Recently, photoacoustic tomography (PAT) has been developed for in vivo sO₂ imaging in live subjects. Specifically, PAT reconstructs three-dimensional images from two-dimensional A-lines obtained by optical-resolution photoacoustic microscopy (OR-PAM).

OR-PAM is a new technique that can obtain an image in vivo of hemoglobin concentration (C_{Hb}) and oxygen saturation with high sensitivity and high resolution. The technique relies on the phenomenon that photoacoustic signals are produced when a pulse of laser light is applied onto a point in a live subject. The laser pulse creates a momentary, tiny, physical expansion of the biological material at the point. The material resiliently restores itself to the original shape and size by releasing the energy as a soundwave. The magnitude of the soundwave depends on the amplitude and wavelength of the light, and absorptivity of the wavelength by the material. By holding the amplitude of the pulse constant, any change in the amplitude of the soundwave can be used to characterize the material.

By selecting wavelengths to which blood components are absorptive, and applying pulses of constant amplitude onto points in a plane that lies across the material, an A-line of the amount of oxyhemoglobin in the plane can be mapped out. A series of A-lines each mapping a deeper region of the material can be assembled into a three-dimensional image of the blood vessels inside the live subject.

However, OR-PAM suffers from the same problem as described above of NIRS and DOT, that the scattering of the incident light pulse by tissue components in the light optical path is greater in the deeper regions of the biological material. In other words, the fluence of the light pulse is reduced more and more as the pulse penetrates deeper into the live subject.

Several methods have been developed to compensate for such fluence loss. One method uses pre-established light diffusion models to estimate fluence attenuation. This method uses an invasive procedure to quantify optical properties of a multilayer skin and tissue, to obtain an empirical estimation of fluence loss on all light travel through tissue. However, living tissues are made of multiple layers of very different components and this approach is too simplistic to be applicable to many types of tissues.

Another approach is to use an iterative method to mathematically estimate the tissue optical properties. This method needs considerable computation time.

Other methods include using reference dyes with known optical properties or a priori knowledge of tissue optical properties, which is invasive and not applicable to certain anatomical sites.

Hence, none of the prior art methods for address image inaccuracy due to fluence loss is satisfactory. It is desirable to propose a method which can improve images obtained by photoacoustic methods of deeper regions of tissue with high fluence loss.

### SUMMARY OF THE INVENTION

In the first aspect, the invention proposes a method of adjusting the quantity of at least one component measured by a photoacoustic monitoring device, comprising the steps of:
a) obtaining n number of photoacoustic responses of n number of components in a sample using n number of pulses of light of a respective wavelength; wherein
   the n number of pulses of light reaching the sample in an optical path; and
   the n number of photoacoustic responses of the component being relatable to the quantity of at least one of the n number of components in the sample;
b) obtaining the photoacoustic response from the sample to another pulse of light,
   the other pulse of light being in a pre-determined reference wavelength;
   the other pulse of light reaching the sample by the same optical path; and
   the other pulse of light reaching the sample in a different time from the to at least one pulse of light;
c) adjusting the quantity of the n number of components by an estimated amount made according to the amplitude of the other pulse of light.

Advantageously, the invention is not limited to monitoring the ratio between two components, such as oxyhemoglobin and deoxyhemoglobin for monitoring blood oxygen saturation. Three or more components can be monitored as long as the absorption coefficients of the components are known, and an additional reference wavelength is used to estimate the loss of fluence of the readings of the other wavelengths.

Preferably, step a) comprises: obtaining two photoacoustic responses of two components in a sample using two pulses of light each of a respective wavelength. More preferably, however, the two components are oxyhemoglobin and deoxyhemoglobin in a sample of living tissue; the quantity of the two components is expressed as blood oxygen saturation.

This feature allows the method to be used to provide three-dimensional images of blood vessel, showing the blood oxygen saturation. Possibly, the blood oxygen saturation is shown in a colour scale (which is not presentable in this black and white specification).

More preferably, the two or more photoacoustic responses are obtained using wavelengths of 532 nm and 558 nm; and the reference wavelength is 545 nm. Even better, wavelengths near the isosbestic points of the components are used, in which case the components are different versions of the same molecules. Therefore, other wavelengths combination can be used to monitor metabolites and so on.

Optionally, the reference wavelength being pre-selected such that loss of light of the reference wavelength in the optical path is useable to estimate the loss of light of the at least one pulse of light; and the estimation for adjusting the at least one photoacoustic response of the at least one component provides that the adjusted photoacoustic response is more accurate after the adjustment.

It is difficult to specify how near should the reference wavelength be to the wavelength of the pulse of light. However, the skilled reader would understand that usually the wavelengths are preferably within the same group, such as ultraviolet, infrared, and so on. Furthermore, the skilled reader should be able find guidance in the principle that the reference wavelength should be selected such that the photoacoustic response of the analyte after normalization should be improved generally.

Typically, the pulses of light are issued from a laser source; the pulses of light are issued at a frequency of 4 kHz and/or with a pulse width of 7 ns.

The invention provides the possible advantage that measurement is non-invasive, and there is possibly no need for prior tissue knowledge, or intensive computation.

In a second aspect, the invention proposes a method of producing a three-dimensional image of blood oxygen saturation, comprising the steps of:
a) directing a light pulse in a first wavelength λ₁ into a point in a biological sample to trigger a first soundwave;
b) measuring the amplitude of the first soundwave;
c) directing at a different time a light pulse in a second wavelength λ₂ into the point in the biological sample to trigger a second soundwave;
d) measuring the amplitude of the second soundwave;
e) directing at another different time a light pulse in a reference wavelength λ₀ into the each point in the plane to trigger a reference soundwave; wherein
   the absorption coefficient of oxyhemoglobin and deoxyhemoglobin in each of the wavelength λ₁, λ₂, λ₀ is known;
f) calculating the blood oxygen saturation based on the following relationship

$$\frac{2 {ε}_{de}^{{λ}_{2}} {ε}_{de}^{{λ}_{1}} {P}_{{λ}_{0}} - {ε}_{de}^{{λ}_{2}} {ε}_{de}^{{λ}_{0}} {P}_{{λ}_{1}} - {ε}_{d e}^{{λ}_{1}} {ε}_{de}^{{λ}_{0}} {P}_{{λ}_{2}}}{2 {ε}_{de}^{{λ}_{2}} {ε}_{de}^{{λ}_{1}} {P}_{{λ}_{0}} - {ε}_{de}^{{λ}_{2}} {ε}_{de}^{{λ}_{0}} {P}_{{λ}_{1}} + {ε}_{o x y}^{{λ}_{2}} {ε}_{de}^{{λ}_{0}} {P}_{{λ}_{1}} - 2 {ε}_{o x y}^{{λ}_{2}} {ε}_{de}^{{λ}_{1}} {P}_{{λ}_{0}}}$$

Where
P_{λ}_{₁} is amplitude of photoacoustic soundwave in wavelength λ₁
P_{λ}_{₂} is amplitude of photoacoustic soundwave in wavelength λ₂
P_{λ}_{₀} is amplitude of photoacoustic soundwave in wavelength λ₀
ε_{oxy}^{λ}^{₁} is the molar extinction coefficient of oxyhemoglobin (HbO₂) in a first wavelength λ₁.
ε_{de}^{λ}^{₁} the molar extinction coefficient of deoxyhemoglobin (HbR) in the first wavelength λ₁;
ε_{oxy}^{λ}^{₂} is the molar extinction coefficient of oxyhemoglobin (HbO₂) in a second wavelength λ₂;
ε_{de}^{λ}^{₂}, the molar extinction coefficient of deoxyhemoglobin (HbR) in the first wavelength λ₂.
ε_{oxy}^{λ}^{₀} is the molar extinction coefficient of oxyhemoglobin (HbO₂) in a second wavelength λ₀;
ε_{de}^{λ}^{₀}, the molar extinction coefficient of deoxyhemoglobin (HbR) in the first wavelength λ₀.
g) repeating above step a) to step h) for every point in a first plane through the biological sample;
h) repeating step g) for a second plane; wherein this second plane is parallel and adjacent parallel to the aforementioned plane.

Preferably, λ₁ is 532 nm, λ₂ is 558 nm; and λ₀ is 545 nm.

Typically, the method is used in optical-resolution photoacoustic microscopy (OR-PAM). Alternatively, the method is used in acoustic-resolution photoacoustic microscopy (AR-PAM).

### BRIEF DESCRIPTION OF THE FIGURES

It will be convenient to further describe the present invention with respect to the accompanying drawings that illustrate possible arrangements of the invention, in which like integers refer to like parts. Other arrangements of the invention are possible, and consequently the particularity of the accompanying drawings is not to be understood as superseding the generality of the preceding description of the invention.

FIG. 1 illustrates an embodiment of the invention;

FIG. 2 illustrates a device that applies the embodiment of FIG. 1;

FIGS. 3(a)-3(c) explain a comparative prior art to the embodiment of FIG. 1;

FIG. 4 illustrates A-lines of the embodiment of FIG. 1;

FIG. 5 illustrates how the embodiment of FIG. 1 is applied and focused onto a point in living tissue for OR-PAM;

FIG. 6 schematically illustrates how the embodiment of FIG. 1 is applied and focused onto a point in living tissue with loss of fluence due to scattering and/or beam broadening of the incident beam;

FIG. 7 illustrate the linearization applied in the embodiment of FIG. 1;

FIG. 8 illustrates isosbestic points in the spectrum of oxyhemoglobin and deoxyhemoglobin, as possibly monitored by the embodiment of FIG. 1;

FIG. 9 illustrates one configuration of the embodiment of FIG. 1;

FIG. 10 illustrates another configuration of the embodiment of FIG. 1;

FIG. 11 shows the time lapse between pulses used in the embodiment of FIG. 7 and FIG. 6;

FIG. 12 illustrates Raman Scattering that may be used in the embodiment of FIG. 7;

FIG. 13 illustrates how the embodiment of FIG. 1 is applied for AR-PAM;

FIG. 14 illustrates the scattering in AR-PAM;

FIG. 15 illustrates improvements as observed in an experiment using an embodiment made according to that of FIG. 1; and

FIG. 16 is a more detailed illustration of the embodiment illustrated in FIG. 10.

### DETAILED DESCRIPTION OF SPECIFIC EMBODIMENTS

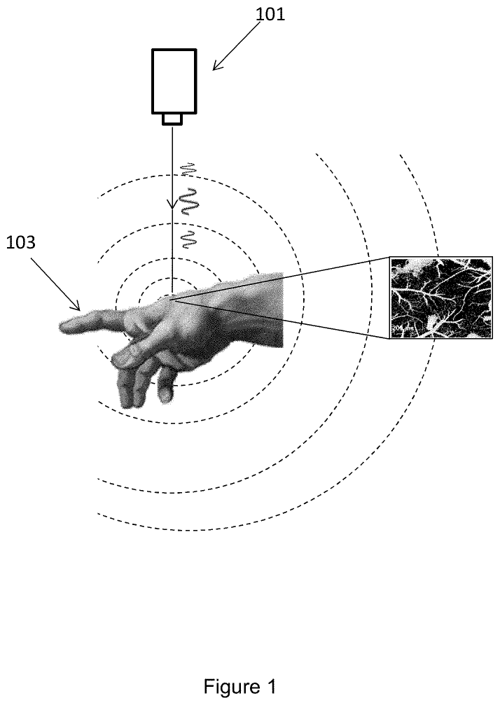

FIG. 1 illustrates a non-ionizing pulse laser 101 applying three pulses of light onto a specific spot inside a living tissue 103, such that inside a hand.

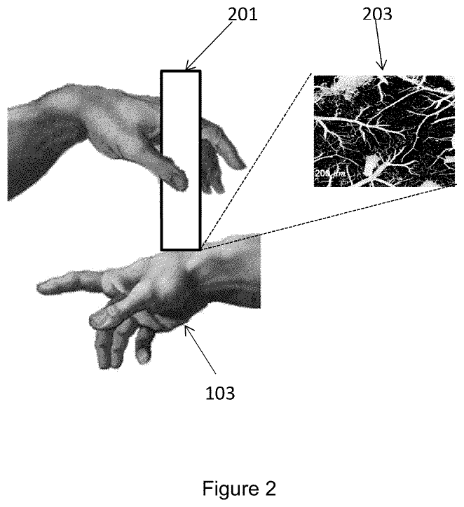

FIG. 2 illustrates a handheld photoacoustic microscope 201 which is configured to apply the three pulses of laser as illustrated in FIG. 1. The microscope comprises a probe which is applied to produce a three-dimensional image of the blood vessels in the living tissue.

To describe the present embodiment effectively, it would be more expedient to explain the prior art in detail first.

### COMPARATIVE PRIOR ART

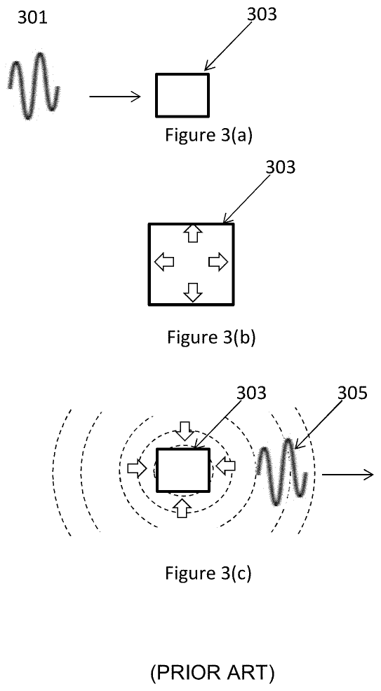

FIGS. 3(a)-(c) illustrates the mechanism of the operation of a photoacoustic microscope according to the prior art.

FIG. 3(a) shows a laser pulse 301 focused onto a very small locality, virtually a point 303, inside a living tissue.

FIG. 3(b) shows how the laser pulse 301 causes the tissue at the focal point to heat up and expand suddenly. As the point 303 cools the expansion reverses. The expansion and contraction cause a pressure wave to propagate through the tissue that can be sensed by a soundwave 305 sensor (not illustrated) coupled directly to it.

Accordingly, FIG. 3(c) shows how as the tiny portion of the tissues relaxes, some of the energy is released in the form of a soundwave 305. This soundwave 305 can be detected by a suitable ultrasound transducer situated appropriately to do so.

Hence, by selecting a wavelength that is more absorbable by blood than other components in human tissue, the amount of blood in the point 303 can be calculated from the magnitude of the soundwave 305 produced. There is no need for calibration to equate the soundwave 305 magnitude to the amount of blood if the absorption coefficient of blood and that wavelength is known in advance.

The absorption coefficient of blood at a wavelength is the absolute amount of absorption of that particular wavelength by a unit quantity of blood, as obtained and established by empirical observation in previous studies. Hence, the soundwave 305 produced by the absorption of an amount of the wavelength is relatable to the amount of blood in the point, by direct multiplication of the absorption coefficient to the magnitude of the soundwave 305.

Even better, instead of using the absorption coefficient of blood, the absorption coefficients of specific blood components, i.e. oxyhemoglobin and deoxyhemoglobin of the particular wavelength, are used to differentiate and calculate these two different components in blood from a single laser pulse. That is, a part of the light is absorbed by oxyhemoglobin and another part by deoxyhemoglobin. However, it is not possible to derive the results of two unknown quantities, i.e. oxyhemoglobin and deoxyhemoglobin, from the single soundwave 305 generated by the absorption of the one specific wavelength. If the absorption coefficient of oxyhemoglobin and the absorption coefficient of deoxyhemoglobin in another wavelength are known, measurements of the resultant soundwave 305 from absorption of the second wavelength in the same point in the living tissue can be made.

Having two measurements of soundwaves 305, each generated by a different wavelength, with known absorption coefficients of oxyhemoglobin and the absorption coefficients of deoxyhemoglobin of both wavelengths, allows one to calculate the amount of oxyhemoglobin and deoxyhemoglobin; it is merely solving two quadratic equations for two unknowns from the two soundwaves 305 amplitudes.

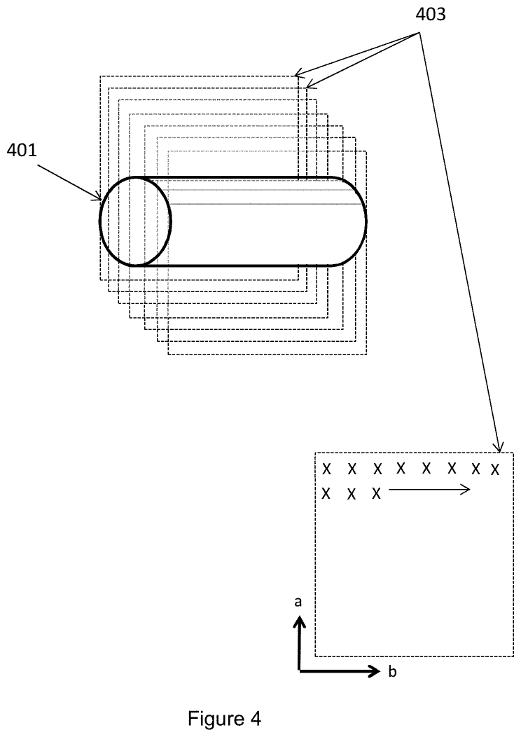

FIG. 4 illustrates how a tomographical image of a blood vessel 401 may be obtained from the inside of living tissues using photoacoustic effects to collect a series of two-dimensional images of the blood vessel, i.e. a series of raster. Typically, as bursts of laser is applied onto discrete points marked X's across the tissue, in a straight line along the a-axis, followed by another line below that line, down along the b-axis, and eventually another one below (not illustrated), the soundwaves 305 provided by each point in that plane in the tissue map out a two-dimensional and cross-sectional image of the plane of tissue. Each plane is known as an A-line 403.

Subsequently, an adjacent parallel plane is mapped out in the same way. When enough A-lines have been mapped out, as illustrated in FIG. 4, the A-lines can be assembled to form a three-dimensional image of the blood vessels in the tissue.

The resolution between the same point in two adjacent planes or A-lines is known as the lateral resolution. However, accuracy of the photoacoustic measurement is affected because the laser pulse is scattered along the way to the focal point. Thus, the deeper the focal point is in the tissue, the more attenuated is the laser pulse. In other words, the fluence of the incident beam is affected by the depth of the focal point.

The prior art illustrated in FIGS. 3(a)-3(c) can be re-expressed mathematically as follows.

The embodiment applies an assumption that the photoacoustic amplitude P_{λ} at a certain wavelength λ is a linear function of an absorption coefficient μₐ^{λ} of the material, such as blood. This may be expressed as

P_{λ}=kFμₐ^{λ},
where
   k is a constant factor related to the pulse amplitude detection sensitivity, and
   F is the local optical fluence.

In other words, the magnitude of the soundwave 305 is directly proportional to the absorption coefficient μₐ^{λ} of the point in the living tissue, after adjusting for change in the fluence of the incident pulse. In this case where the material is blood, this means that the more concentrated the blood, the more the specific wavelength λ is absorbed by the blood to produce a proportionally greater burst of soundwave 305, P_{λ}.

The relationship between concentration of blood and the photoacoustic amplitude as defined by the absorption coefficient μₐ^{λ} is assumed to be certain, and the photoacoustic amplitude as measured can be used directly to calculate the amount of blood.

As the two main components in blood are oxyhemoglobin and deoxyhemoglobin, the absorption coefficient μₐ^{λ} of blood of any given wavelength is the resultant absorption of the wavelength by these two components. The absorption coefficient of oxyhemoglobin and deoxyhemoglobin at one wavelength can be written as follows.

μₐ^{λ}=ρC_{HbT}[sO₂ε_{oxy}^{λ}+(1−sO₂)ε_{de}^{λ}],
where
   ρ is a known constant coefficient;
   C_{HbT} is the total hemoglobin concentration;
   ε_{oxy}^{λ} is the molar extinction coefficient of oxyhemoglobin (HbO₂);
   ε_{de}^{λ} is the molar extinction coefficient of deoxyhemoglobin (HbR).

As expressed above, blood oxygen saturation sO₂ is expressed as related to the absorption coefficient μₐ^{λ}, the molar extinction coefficients of oxyhemoglobin (HbO₂) ε_{oxy}^{λ}, and to the molar extinction coefficients of deoxyhemoglobin (HbR) Σ_{de}^{λ}.

A molar extinction coefficient is a measurement of how strongly a chemical species reduces the intensity of light of a given wavelength, typically by absorbing the light. It is an intrinsic property of the species and does not require elaboration.

If a pulse of laser of a wavelength that is absorbed by blood is used to create a photoacoustic soundwave 305 in a certain point in a piece of tissue, the magnitude of the soundwave 305 can be used to determine the amount of blood present in that point by multiplying it with the absorption coefficient μₐ^{λ} of blood in that wavelength. However, it is not possible to tell how much of the blood is oxyhemoglobin and how much is deoxyhemoglobin. To find the answer to these two unknowns, a pulse of laser of another wavelength that is absorbed by blood is used to create another photoacoustic soundwave 305. The magnitude of the soundwave 305 produced by the photoacoustic effect of the same point in the tissue in this other wavelength can also be used to calculate the total amount of blood in the point.

As the two absorption coefficients μₐ^{λ}^{₀} and μₐ^{A}^{₁} each represents the amount of light of respective wavelengths λ₀, λ₁, that is absorbed by blood, the amplitudes of the soundwave 305 produced by each wavelength can be solved as a quadratic equation.

P_{λ}_{₁}=kFμₐ^{λ}^{₁} (1)

P_{λ}_{₂}=kFμₐ^{λ}^{₂} (1)

The above quadratic equations can be expanded and solved for the amount of oxyhemoglobin and deoxyhemoglobin, which make up the composite value of blood oxygen saturation (sO₂).

P_{λ}_{₁} is obtained experimentally by emitting a pulse of wavelength λ₁ into the point in the tissue, and P_{λ}_{₂} is also obtained experimentally by emitting a pulse of wavelength λ₂ into the point in the tissue.

μₐ^{λ}^{₁} and μₐ^{λ}^{₂} are defined follows

μₐ^{λ}^{₁}=ρC_{HbT}[sO₂ε_{oxy}^{λ}^{₁}+(1−sO₂)ε_{de}^{λ}^{₁}] (3)

μₐ^{λ}^{₂}=ρC_{HbT}[sO₂ε_{oxy}^{λ}^{₂}+(1−sO₂)ε_{de}^{λ}^{₂}] (4)

Typically, ε_{oxy}^{λ}^{₁} the molar extinction coefficients of oxyhemoglobin (HbO₂); and ε_{de}^{λ}^{₁}, the molar extinction coefficients of deoxyhemoglobin (HbR) in the first wavelength λ₁ are known.

Furthermore, ε_{oxy}^{λ}^{₂}, the molar extinction coefficients of oxyhemoglobin (HbO₂); and ε_{de}^{λ}^{₂} the molar extinction coefficients of deoxyhemoglobin (HbR) in the second wavelength λ₂ are known.

In practice, however, it is possible that μₐ^{λ}^{₁} land μₐ^{λ}^{₂} as well as C_{HbT} are not known, and sO₂ can be still calculated just by solving equation 3 and equation 4.

Upon expansion of equation 1 and equation 2 the amount of oxyhemoglobin (HbO₂) and deoxyhemoglobin (HbR) can be obtained by solving the quadratic equation.

Re-arranging the quadratic equations gives the following formula to obtain sO₂ from light pulses of two different wavelengths:

$${s O}_{2} = \frac{{P}_{{λ}_{1}} {ε}_{d e}^{{λ}_{2}} - {P}_{{λ}_{2}} {ε}_{d e}^{{λ}_{1}}}{{P}_{{λ}_{1}} ( {ε}_{d e}^{{λ}_{2}} - {ε}_{o x y}^{{λ}_{2}} ) - {P}_{{λ}_{2}} ( {ε}_{d e}^{{λ}_{1}} - {ε}_{o x y}^{{λ}_{1}} )} \qquad ( 5 )$$

### The Present Embodiment

Equation 5 is used in the prior art. In the prior art, the effect of loss of fluence by light scattering of the incident pulse is not addressed.

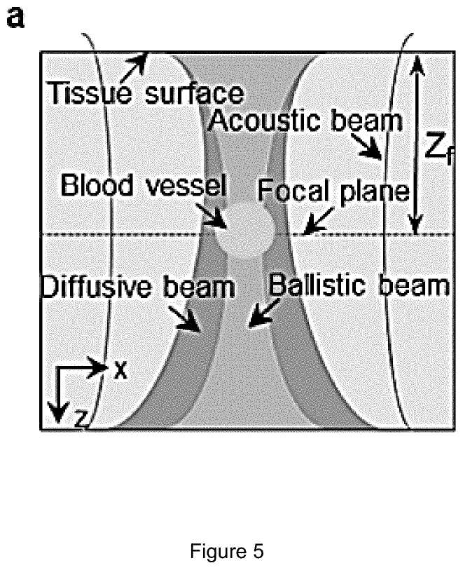

FIG. 5 illustrates how a ballistic pulse (incident pulse) passing through a blood in the z-axis direction vessel but having a focal point on the blood vessel creates an acoustic beam in the orthogonal direction of the direction of travel of the ballistic beam, or x-axis. Beyond the point of focus, the light beam becomes out of focus and diffuses into the tissue.

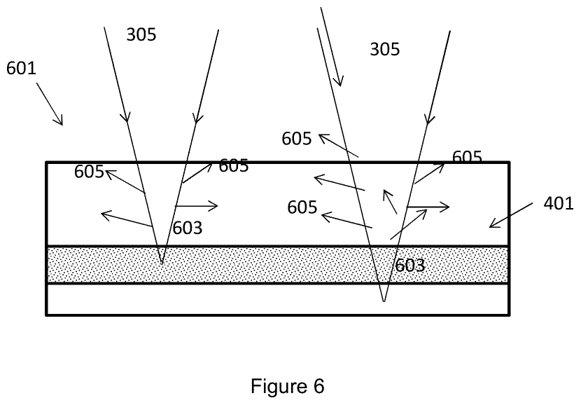

FIG. 6 shows how, when the ballistic pulse light enters the skin 601 of a living tissue, such as that of the hand shown in FIG. 1, and traverse towards the focal point 603, part of the light is lost through being scattered, at 605, by substance in the optical pathway. Along the way to the point, part of the fluence of the beam is lose due to scattering of the beam. The left side of the drawing shows a beam focused onto a blood vessel with a certain amount of scattering, and therefore fluence loss, which results in a smaller amplitude in the final soundwave 305. Therefore, in the prior art, the smaller amplitude would be misinterpreted to be due to a smaller level blood oxygen saturation. The right side of the drawing shows a beam focused much deeper than the example on the left side, and this means the trajectory of the light pulse had to penetrate deeper into the tissue before the light pulse is focused, which results even more loss of fluence due to more scattering. Therefore, the amplitude in the final soundwave 305 is even smaller.

The extent of scattering and back-scattering along the optical path to every localized point in sample is different for different wavelengths. The effect of loss of fluence due to scattering of the incident laser pulse, and how the reference wavelength may be used to normalize the A-line images may be expressed mathematically as follows. The soundwave produced by the reference wavelength in the same point in the tissue can be measured directly.

The following expression shows the relationship between the amplitude of the soundwave from one acoustic voxel and wavelength.

$${P}_{λ} = \int {k}_{0} exp ( - \frac{2 {r}^{2}}{{w}^{2}} ) Γ η {F}_{λ} ( r ) {μ}_{a}^{λ} ( r ) d r \qquad ( 6 )$$

where
   k₀ exp

$$( - \frac{2 {r}^{2}}{{w}^{2}} )$$

is the acoustic detection sensitivity,
k₀ is the peak sensitivity at the center of the acoustic beam,
w is the characteristic radius of the acoustic beam,
Γ is the local Grueneisen parameter,
η is the photothermal conversion efficiency,
F_{λ}(r) is the local optical fluence at position r,
μₐ^{λ}(r) is the absorption coefficient of a substance.

Fluence F_{λ} is a function of the wavelength λ. Different wavelengths suffer from different extent of scattering on reaching the point of focus, and therefore different extent of attenuation.

Assuming that μₐ^{λ}(r) is uniform within the analysis object O(r),

μₐ^{μ}(r)=μₐ^{λ}O(r).

In the present case, the object is blood in the tissue onto which the laser pulse is focused.

In present embodiment provides the possibility of adjusting for inaccuracy in the above model by using a third, reference, wavelength to normalize the sO₂ obtained by the above method.

The extent of the loss of fluence due to scattering of the ballistic pulse is wavelength-dependent; some wavelengths penetrate some materials better than other wavelengths.

However, as human tissue has too many different components in the optical path of the laser, such that it is not possible to provide a theoretical model that applies to tissues of all test subjects. Therefore, embodiment proposes using empirical observations to adjust for the loss in fluence. More specifically, the embodiment proposes measuring the amplitude of a pulse of a reference wavelength to estimate the fluence loss of the pulses of the wavelengths used to measure sO₂. That is, fluence of the reference wavelength may be used to estimate fluence loss of each of the first wavelength and the second wavelength, if the wavelengths are within a narrow range in the spectrum.

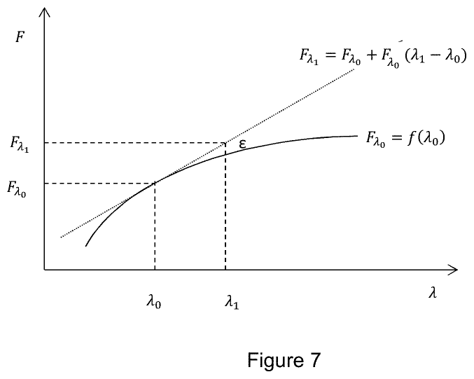

One possible method of estimating the fluence loss in the first wavelength and the second wavelength is to extrapolate from the fluence loss in the reference wavelength, by assuming that fluence loss is linearly wavelength dependent. FIG. 7 explains the concept of the linearization as applied in the present embodiment. The x-axis represents wavelength while the y-axis represents the theoretical local fluence of the corresponding wavelength in living tissue. The straight-line FIG. 7 is the first derivative of the curve of the function F=f(λ). The straight line is a tangent on the point of the curve read at wavelength λ₀, the reading being F_{λ}_{₀}. Where it is not possible to calculate or observe the value of F_{λ} at wavelength λ, it is possible to estimate F_{λ} by assuming that F_{λ} and F_{λ}_{₀} are points on the linear tangent. There could be an error, as marked by E. However, if the two wavelengths are very near, the error is small. In this way, empirical observation of the fluence loss of the reference wavelength on reaching a point in the tissue can be used to estimate the fluence loss of each of the second wavelength and reference wavelength.

Therefore, one way of expressing the estimated fluence F_{λ} of wavelength λ, is to add to the fluence F_{λ}_{₀} of the reference wavelength the derivative F′_{λ}_{₀} of the reference wavelength across the difference (λ−λ₀).

F_{λ}=F_{λ}_{₀}+F_{λ}_{₀}(λ−λ₀)
where
F′_{λ}_{₀} represents the first derivative of the fluence to the optical wavelength; and
F′_{λ}_{₀}(λ−λ₀) is the change in fluence between wavelengths λ₀ and λ.

Accordingly, in a reasonably narrow spectrum, the fluence or, more specifically, loss of fluence can be approximated from the reference wavelength λ₀, i.e. by linearizing from the soundwave 305 produced by the reference wavelength λ₀, provided that the difference between λ₀ and λ₁ is not too large. If one wavelength is too far from the other wavelength along the electromagnetic spectrum, the absorbance, reflectance or scattering coefficient of components in the optical path could be too different for the assumption to be valid.

In biological tissues, optical scattering is usually much higher than absorption, and the scattering coefficient can be approximate as a low-order polynomial function of the optical wavelength. Thus, when the spectrum is narrow, the linearized local fluence can be a good approximation of the true value.

With linearization, Equation 6 becomes

$${P}_{λ} = {μ}_{a}^{λ} \int {k}_{0} exp ( - \frac{2 {r}^{2}}{{w}^{2}} ) Γ η [ {F}_{{λ}_{0}} ( r ) + {F}_{{λ}_{0}}^{′} ( r ) ( λ - {λ}_{0} ) ] O ( r ) dr \qquad ( 7 )$$

Denote

$${K}_{1} = \int {k}_{0} exp ( - \frac{2 {r}^{2}}{{w}^{2}} ) Γη {F}_{{λ}_{0}} ( r ) O ( r ) dr$$

$${K}_{2} = \int {k}_{0} exp ( - \frac{2 {r}^{2}}{{w}^{2}} ) Γη {F}_{{λ}_{0}}^{′} ( r ) O ( r ) dr$$

Note that K₁ and K₂ are independent of the optical wavelength.

Then Equation 7 becomes

P_{λ}=μₐ^{λ}[K₁+K₂(λ−λ₀)]. (8)

Here, the photoacoustic amplitude becomes a product of the optical absorption coefficient and a linear function of the optical wavelength.

Substituting μₐ^{λ}=ρC_{HbT}[sO₂ε_{oxy}^{λ}+(1−sO₂)ε_{de}^{λ}] into Equation 8), the photoacoustic amplitude becomes

P_{λ}=[sO₂ε_{oxy}^{λ}+(1−sO₂)ε_{de}^{λ}][K̅₁̅+K̅₂̅(λ−λ₀)], (9)
Where

K̅₁̅=K₁ρC_{HbT},

K̅₂̅=K₂ρC_{HbT}

K̅₁̅ and K̅₂̅ which were obtained for λ₀ but now assumed to be constant for all wavelengths.

To solve sO₂, laser pulses in three wavelengths (λ₀, λ₁ and λ₂) are directed into the same point inside the tissue, obtaining P_{λ}_{₀}, P_{λ}_{₁} and P_{λ}_{₂}. A preferred choice of wavelengths in one specific embodiment is λ₀=545 nm, λ₁=532 nm, and λ₂=558 nm.

Generally, however, it has been found that for wavelengths shorter than 610 nm, the prediction error is less than 0.3%. The prediction error is as high as 13% at 779 nm. So the range of narrow spectrum is about 532 to 800 nm.

λ₀ is the reference wavelength. Therefore, denoting Δλ=λ₂−λ₀=−(λ₁−λ₀), the three photoacoustic amplitudes can be written as follow.

P_{λ}_{₀}=[sO₂ε_{oxy}^{λ}^{₀}+ε_{de}^{λ}^{₀}−sO₂ε_{de}^{λ}^{₀}][K̅₁̅] (10)

P_{λ}_{₁}=[sO₂ε_{oxy}^{λ}^{₁}+ε_{de}^{λ}^{₁}−sO₂ε_{de}^{λ}^{₁}][K̅₁̅−K̅₂̅Δλ] (11)

P_{λ}_{₂}=[sO₂ε_{oxy}^{λ}^{₁}+(1−sO₂)ε_{de}^{λ}^{₂}][K̅₁̅+K̅₂̅Δλ] (12)

As λ₀ and λ₁ are close to isosbestic points, the equation can be simplified by assuming ε_{oxy}^{λ}^{₀}=ε_{de}^{λ}^{₀}, ε_{oxy}^{λ}^{₁}=ε_{de}^{λ}^{₁}, and the equations can be re-written into the following.

P_{λ}_{₀}=ε_{de}^{λ}^{₀}K̅₁̅ (13)

P_{λ}_{₁}=ε_{de}^{λ}^{₁}(K̅₁̅−K̅₂̅Δλ) (14)

P_{λ}_{₂}=[sO₂ε_{oxy}^{λ}^{₂}+(1−sO₂)ε_{de}^{λ}^{₂}](K̅₁̅+K̅₂̅Δλ) (15)

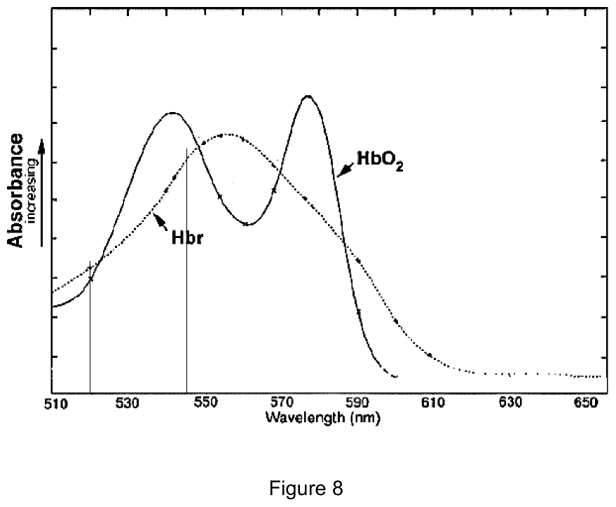

FIG. 8 shows the overlapping absorption spectra of oxyhemoglobin and deoxyhemoglobin. The vertical lines in the chart cut across two, amongst others, isosbestic points in the overlapping spectra. That is, the absorbance of oxyhemoglobin and the absorbance of deoxyhemoglobin of a wavelength at an isosbestic point are equal. The skilled reader would appreciate that choice of wavelengths near isosbestic points are convenient for simplifying the equations, but are not necessary.

Combining Equation 13, Equation 14 and Equation 15, sO₂ can be calculated as

$${s O}_{2} = \frac{2 {ε}_{de}^{{λ}_{2}} {ε}_{de}^{{λ}_{1}} {P}_{{λ}_{0}} - {ε}_{de}^{{λ}_{2}} {ε}_{de}^{{λ}_{0}} {P}_{{λ}_{1}} - {ε}_{d e}^{{λ}_{1}} {ε}_{de}^{{λ}_{0}} {P}_{{λ}_{2}}}{2 {ε}_{de}^{{λ}_{2}} {ε}_{de}^{{λ}_{1}} {P}_{{λ}_{0}} - {ε}_{de}^{{λ}_{2}} {ε}_{de}^{{λ}_{0}} {P}_{{λ}_{1}} + {ε}_{o x y}^{{λ}_{2}} {ε}_{de}^{{λ}_{0}} {P}_{{λ}_{1}} - 2 {ε}_{o x y}^{{λ}_{2}} {ε}_{de}^{{λ}_{1}} {P}_{{λ}_{0}}} \qquad ( 16 )$$

Equation 16 allows blood oxygen saturation measured by the first wavelength and the second wavelength to be adjusted by observation in the reference wavelength for improved accuracy.

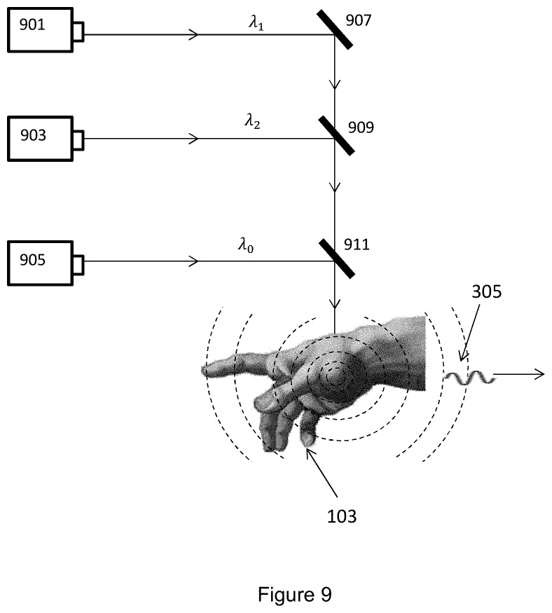

FIG. 9 illustrates the structure of a photoacoustic microscope according to an embodiment of the invention. In the embodiment of FIG. 8, three pulses, each in a different wavelength, λ₀, λ₁, λ₂ are used to trigger a photoacoustic response in a point location in the living tissue. Each of the pulses is issued by a separate laser 901, 903, 905.

The pulses are focus onto the same point inside the living tissue. The first pulse λ₀ is issued and hits the reflective side of a beam splitter 911 to be directed to the living tissue. The focusing devices such as lenses are not illustrated for clarity of the illustration. Subsequently, the second pulse λ₂ is issued and hits the reflective side of another beam splitter 909 to be directed through the first beam splitter 911 to the living tissue 103. Finally, the third pulse λ₃ is issued and hits a mirror 907 to be directed through the first beam splitter 911 and second beam splitter 909 to the living tissue 103.

Although FIG. 9 is shown schematically, the skilled man would understand that the arrangement of the optical devices is such that all the wavelengths travel in the same optical path within the tissue, and has the same focal point.

The pulses are issued one after another and arrive at the focal point in different times, so that the soundwaves 305 generated by blood in the point, in response to each wavelength, may be identified as having trigger by which wavelength. Using the above mentioned formula, the sO₂ level can be estimated.

Preferably, the laser a nanosecond pulse later, and the pulse frequency is 4 kHz, and the preferred pulse width is about 7 ns. However, the preferred pulse repetition rate can be with the 0˜1 MHz. The pulse width can be any width within the range of 2˜10 ns.

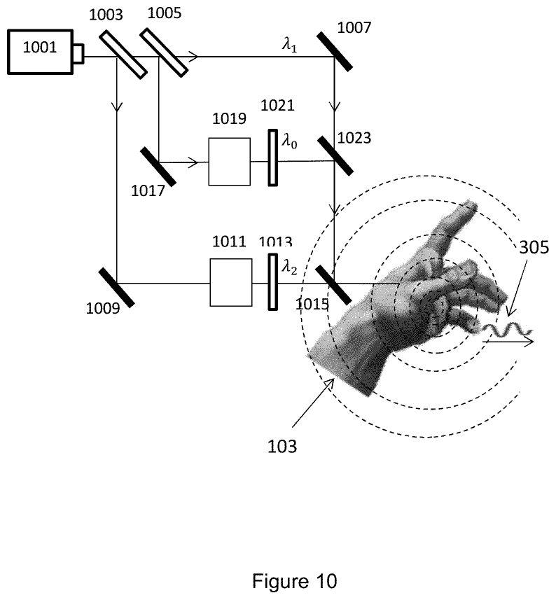

FIG. 10 illustrates the structure of a photoacoustic device according to another embodiment of the invention, in which there is only one source of light 1001 in a single wavelength. A laser pulse is issued from the light source, and is split by a first polarizing beam splitter 1003 into two division pulses. One of the division pulses is directed to yet a second polarizing beam splitter 1005 to be further divided into two division pulses. Therefore, there are now three pulses split from the original pulse.

The pulse that continues to traverse the original incident optical path, λ₁, which is the pulse has passed through the two polarizing beam splitters, 1003, 1005 is directed to a first mirror 1007 that will reflect and focus the pulse into the living tissue 103, i.e. the first pulse. The wavelength of this pulse is the same as that issued by the laser source 1001.

The second one of the three pulses, λ₂, is split from the original pulse by the first polarizing beam splitter 1003 directed to a second mirror 1009 that will reflect the pulse into a 100 metre graded-index multi-mode fibre 1011. Stimulated Raman scattering effect occurs when the optical beam pass through the fiber 1011. The longer wavelength λ₂ will be generated through the stimulated-Raman-scattering (SRS) effect. Upon exiting the optic fibre 1011 the pulse passes to a short-pass filter 1013 is placed after the optic fibre 1011. However, the short-pass filter 1013 will only allow the part of the second pulse that comprises a wavelength shifted from the original wavelength by Raman Effect into becoming a longer wavelength to pass through. The second pulse then passes through a dichroic mirror 1015 that gives a pre-selected range of wavelengths to passage. The length of the optic fibre 1011 is selected such that refraction effect in the optic fibre delays propagation of the pulse such that the second pulse reaches the living tissue 103 after the first pulse had arrived.

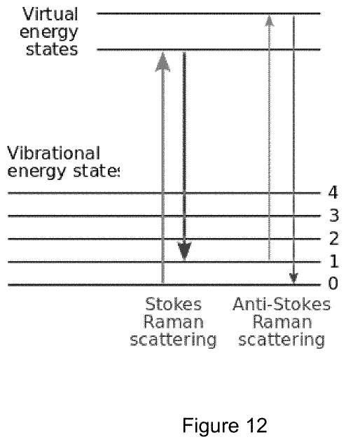

Also, the skilled man would know that the Raman wavelengths derived from the pulse of the original wavelength can be up to five wavelengths, which is dependent on the maximum energy of the laser. Any of these five wavelengths can be used in the embodiment. FIG. 12 (taken from www.wikipedia.org) explains the stimulated Raman scattering of wavelengths. Raman scattering is the effect of a laser exciting a material such that the material re-emits the laser in a lower or higher wavelength. This shift to a greater wavelength is called a Stokes shift, a shift to a narrower wavelength is called an anti-Stokes shift. The fraction of photons by the Raman effect is approximately 1 in 10 million). The magnitude of the Raman effect in a material correlates with polarizability of the electrons in a molecule of the material. Materials made of molecules with relatively neutral bonds (e.g. C—C, C—H, C═C) are strong Raman scatterers.

The third one of the three pulses is directed from the second polarizing beam splitter 1005 to a second mirror 1017 that will reflect the pulse into another optic fibre 1019, this time a 30-metre-long polarising-maintaining single-mode. Upon exiting the optic fibre 1019, the part of the pulse that has a wavelength that has been shifted by Raman Effect into becoming a longer wavelength passes through a long-pass filter 1021 to a beam splitter 1023, to be reflected to the dichroic mirror 1015. The beam splitter 1023 is used as a mirror here so that the optical path of this third pulse can become coincident with the optical path of the other pulses. The dichroic mirror 1015 simply reflects the third pulse to focus onto the living tissue. Again, the length of 30 m other optic fibre is selected such that refraction effect in the optic fibre delays propagation of the pulse, and third pulse reaches the living tissue 103 before the second pulse had arrived.

The skilled man would understand that the arrangement of the optical devices in FIG. 10 is such that all the wavelengths travel in the same optical path within the tissue, and has the same focal point.

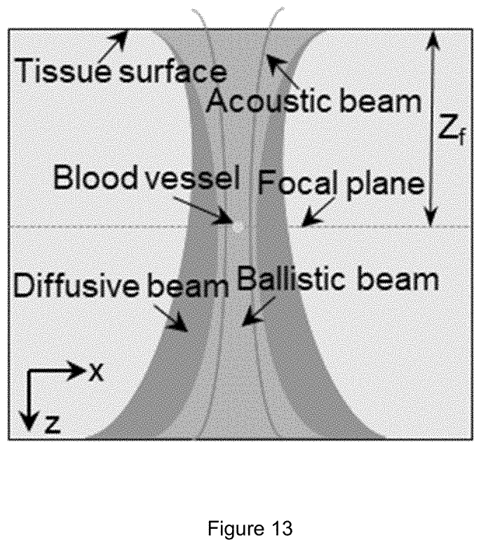

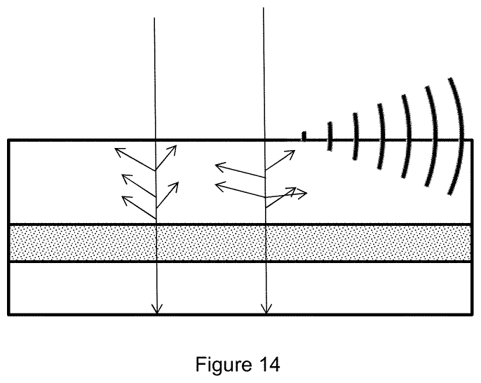

The described embodiments have been described with optical resolution photoacoustic microscopy. However, the embodiments can be applied to other types of photoacoustic microscopy. FIGS. 13 and 14 show another type of photoacoustic microscopy, in which the pulse of light is not focused onto different points in the tissue to build the A-lines but penetrates the tissue more evenly compared to OR-PAM. The positions of the blood in blood vessels are determined instead by resolving the position of the soundwaves 305. This is known as acoustic resolution photoacoustic microscopy (AR-PAM). Comparing FIG. 5 and FIG. 13, it can be seen that the drawings show that, in OR-PAM, the acoustic beam size is larger than the optical beam, whereas in AR-PAM, the optical beam is larger than the acoustic beam size. Therefore, the above embodiments can be applied to acoustic resolution photoacoustic microscopy to reduce loss of fluence due to scattering.

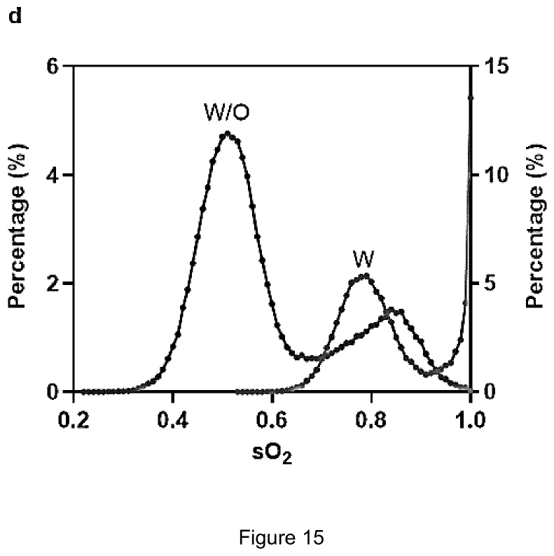

FIG. 15 illustrates improvements as observed in an experiment using an embodiment made using in OP-PAM. W/O indicates the sO2 level without normalization and W indicates that the sO2 level has become higher after normalization, which has been found to be more accurate.

### EXAMPLES OF APPLICATIONS OF EMBODIMENTS

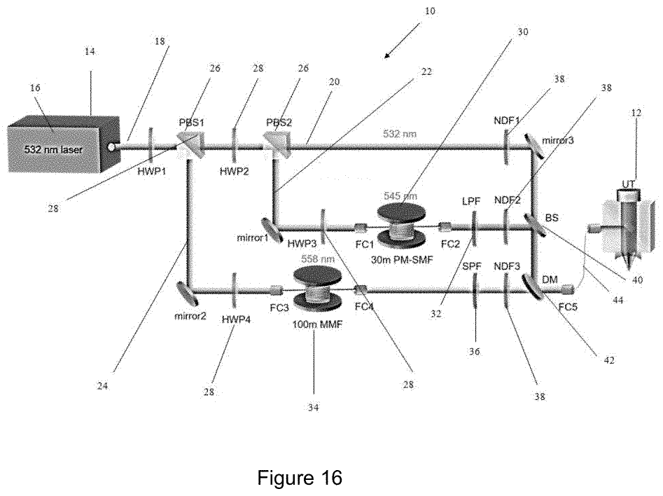

FIG. 16 illustrates the embodiment of FIG. 10 in greater detail, which is used to obtain experimental results as discussed further on in this description.

FIG. 16 shows components of an optical-resolution photoacoustic microscopy (OR-PAM) system 10 having an ultrasonic transducer (UT) 12 connected to a laser system 14, which includes numerous optical components between the laser system 14 and the UT 12. The system in the example outputs 3 wavelengths although the system can be configured to output and process signals from 3 or more wavelengths.

The laser system 14 comprises a nanosecond pulsed laser 16 configured as a pump laser, which in the present example is a 532-nm wavelength laser of the type VPFL-G-20 from SpectraPhysics®. The laser is configured to emit pulses at a repetition rate of 4 kHz, with a pulse width of 7 ns. The pump beam 18 from the laser is split into three optical paths, a 532-nm direct path 20, a 545-nm Raman path 22, and a 558-nm Raman path 24 via two polarizing beam splitters 26, namely PBS1 and PBS2. The pulse energies of the three paths 20, 22, 24 are adjusted by half-wave plates 28, shown as HWP1, HWP2, HWP3 and HWP4.

In the 545-nm Raman path 22, a 30-m polarization-maintaining single-mode fibre 30 e.g. PM-SMF, PM-S405-XP from NUFERNR™ is used to generate a 545-nm wavelength pulse through the stimulated-Raman-scattering (SRS) effect. Half-wave plate HWP3 28 is rotatable to adjust the polarization of the incident light so that the pulse energy of the 545-nm wavelength is maximized. A long-pass filter 32 e.g. LPF, T540lpxr, from CHROMAR™ is placed after the fibre 30 to pass the 545-nm wavelength and reject the 532-nm wavelength.

In the 558-nm Raman path 24, a 100-m graded-index multi-mode fibre 34 e.g. MMF, GIMMSC (50/125) HT from FIBERCORER™ is used to generate the 558-nm wavelength through the SRS effect. The pulse energy of the 558-nm wavelength can be maximized via rotating the half-wave plate HWP4 28 to adjust the polarization of the incident light. Wavelengths longer than 570 nm are rejected by a short pass filter 36 e.g. SPF, RPE570SP from OMEGAR™. The pulse energies of the three wavelengths can be adjusted by a variable neutral density filter 38 e.g. NDC-50C-2, from Thorlabs Inc® in each path 20, 22, 24—three neutral density filters 38 are provided in each path, respectively, NDF1, NDF2 and NDF3. A 10/90 beam splitter (BS) 40 combines the direct 532-nm wavelength with the Raman 545-nm wavelength. A 550-nm long-pass dichroic mirror 42 e.g. DM, T550lpxr0UF1 from CHROMAR™ is used to combine the 532/545-nm wavelengths with the 558-nm wavelength. At the last stage, the three wavelengths are coupled into an OR-PAM probe via a 2-meter single-mode fibre 44 e.g. P1-460B-FC-2 from Thorlabs Inc®. The time delay of the two paths can be measured using a photodiode.

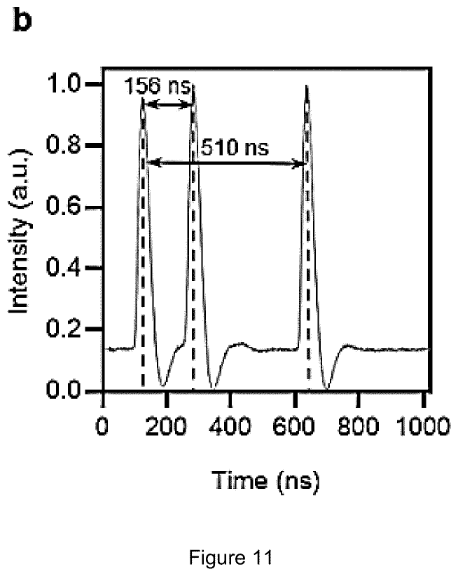

FIG. 11 illustrates the time delays among different pulses due to the delay in fibres and free space, wherein the time delays for the 2^{nd} (545 nm) and the 3^{rd} (558 nm) pulses are 156 and 510 ns. At each scanning spot, the OR-PAM system 10 acquires profiles e.g. A-lines of each of the three wavelengths, sequentially. Volumetric images are acquired via raster scanning using the UT 12. Alternatively, each wavelength can be derived from a different source and collectively configured to emit pulses having the timing and intensity as per FIG. 1b.

The self-fluence-compensation method is demonstrated in functional brain imaging. The protocol of animal experiments was approved by the animal ethical committee of the City University of Hong Kong. PA images of the mouse brain are acquired at 532 nm, 545 nm, and 558 nm. For each wavelength, the pulse energy is about 70 nJ, the pulse repetition rate is 4 kHz, and 700×700 A-lines are acquired for 3D imaging. The step size in the lateral direction is 2.5 μm.

The arterial sO₂ peaks without and with fluence compensation are 0.85 and 0.99, respectively. The venous sO₂ peaks without and with fluence compensation are 0.52 and 0.81, respectively, as shown in FIG. 15, where W/O indicates the chart without compensation and W indicates the chart that has compensation.

The self-compensated arterial sO2 values are in the range of 0.95˜0.99, and the compensated venous sO2 values are over 0.80, which are consistent with normal physiological values.

It is observed that in both the arteries and the veins, sO₂ is corrected more in the distal end than that in the root end of the vessels. For example, along the arrow direction of an artery, the vessel diameter gradually decreases, and the sO₂ improvement becomes more obvious.

It has also been found that in both the artery and the vein, the sO₂ improvement is bigger in the smaller vessel segments, which is consistent with the numerical simulation results.

The described technique can be used in onto non-living material is within the scope of this application, such as for analysis of wood, leather material and tissues in material or archaeology studies.

Furthermore, it is possible to apply the technology to detect only one component in tissue, such as sugars or proteins, in which case only one wavelength is needed to measure the amount of the component used with the reference wavelength to adjust the readings.

The skilled man would understand that the choice of a linear model is just an option. Beside the linear model, any skewed model can be used to modify the photoacoustic readings in the first wavelength and the second wavelength.

While there has been described in the foregoing description preferred embodiments of the present invention, it will be understood by those skilled in the technology concerned that many variations or modifications in details of design, construction or operation may be made without departing from the scope of the present invention as claimed.

1. A method of adjusting the quantity of at least one component measured by a photoacoustic monitoring device, comprising the steps of:
d) obtaining n number of photoacoustic responses of n number of components in a sample using n number of pulses of light of a respective wavelength; wherein
the n number of pulses of light reaching the sample in an optical path; and
the n number of photoacoustic responses of the component being relatable to the quantity of at least one of the n number of components in the sample;
e) obtaining the photoacoustic response from the sample to another pulse of light,
the other pulse of light being in a pre-determined reference wavelength;
the other pulse of light reaching the sample by the same optical path; and
the other pulse of light reaching the sample in a different time from the to at least one pulse of light;
f) adjusting the quantity of the n number of components by an estimated amount made according to the amplitude of the other pulse of light.

2. A method of adjusting the quantity of a component measured by a photoacoustic monitoring device as claimed in claim 1, wherein
step a) comprises:
obtaining two photoacoustic responses of two components in a sample using two pulses of light each of a respective wavelength.

3. A method of adjusting the quantity of a component measured by a photoacoustic monitoring device as claimed in claim 1, wherein
two components are oxyhemoglobin and deoxyhemoglobin in a sample of living tissue;
the quantity of the two components is expressed as blood oxygen saturation.

4. A method of adjusting the quantity of a component measured by a photoacoustic monitoring device as claimed in claim 3, wherein
the two or more photoacoustic responses are obtained using wavelengths of 532 nm and 558 nm; and
the reference wavelength is 545 nm.

5. A method of adjusting the quantity of a component measured by a photoacoustic monitoring device as claimed in claim 2, wherein
the reference wavelength being pre-selected such that loss of light of the reference wavelength in the optical path is useable to estimate the loss of light of the at least one pulse of light; and
the estimation for adjusting the at least one photoacoustic response of the at least one component provides that the adjusted photoacoustic response is more accurate after the adjustment.

6. A method of adjusting the quantity of a component measured by a photoacoustic monitoring device as claimed in claim 1, wherein
the pulses of light are issued from a laser source;
the pulses of light issued at a frequency of 4 kHz and/or with a pulse width of 7 ns.

7. A method of producing a three-dimensional image of blood oxygen saturation, comprising the steps of:
i) directing a light pulse in a first wavelength λ₁ into a point in a biological sample to trigger a first soundwave;
j) measuring the amplitude of the first soundwave;
k) directing at a different time a light pulse in a second wavelength λ₂ into the point in the biological sample to trigger a second soundwave;
l) measuring the amplitude of the second soundwave;
m) directing at another different time a light pulse in a reference wavelength λ₀ into the each point in the plane to trigger a reference soundwave; wherein
the absorption coefficient of oxyhemoglobin and deoxyhemoglobin in each of the wavelength λ₁, λ₂, λ₀ is known;
n) calculating the blood oxygen saturation based on the following relationship
$$\frac{2 {ε}_{de}^{{λ}_{2}} {ε}_{de}^{{λ}_{1}} {P}_{{λ}_{0}} - {ε}_{de}^{{λ}_{2}} {ε}_{de}^{{λ}_{0}} {P}_{{λ}_{1}} - {ε}_{d e}^{{λ}_{1}} {ε}_{de}^{{λ}_{0}} {P}_{{λ}_{2}}}{2 {ε}_{de}^{{λ}_{2}} {ε}_{de}^{{λ}_{1}} {P}_{{λ}_{0}} - {ε}_{de}^{{λ}_{2}} {ε}_{de}^{{λ}_{0}} {P}_{{λ}_{1}} + {ε}_{o x y}^{{λ}_{2}} {ε}_{de}^{{λ}_{0}} {P}_{{λ}_{1}} - 2 {ε}_{o x y}^{{λ}_{2}} {ε}_{de}^{{λ}_{1}} {P}_{{λ}_{0}}}$$
Where
P_{λ}_{₁} is amplitude of photoacoustic soundwave in wavelength λ₁
P_{λ}_{₂} is amplitude of photoacoustic soundwave in wavelength λ₂
P_{λ}_{₀} is amplitude of photoacoustic soundwave in wavelength λ₀
ε_{oxy}^{λ}^{₁} is the molar extinction coefficient of oxyhemoglobin (HbO₂) in a first wavelength λ₁.
ε_{de}^{λ}^{₁}, the molar extinction coefficient of deoxyhemoglobin (HbR) in the first wavelength λ₁;
ε_{oxy}^{λ}^{₂} is the molar extinction coefficient of oxyhemoglobin (HbO₂) in a second wavelength λ₂;
ε_{de}^{λ}^{₂}, the molar extinction coefficient of deoxyhemoglobin (HbR) in the first wavelength λ₂.
ε_{oxy}^{λ}^{₀} is the molar extinction coefficient of oxyhemoglobin (HbO₂) in a second wavelength λ₀;
ε_{de}^{λ}^{₀}, the molar extinction coefficient of deoxyhemoglobin (HbR) in the first wavelength λ₀.
o) repeating above step a) to step h) for every point in a first plane through the biological sample;
p) repeating step g) for a second plane; wherein this second plane is parallel and adjacent parallel to the aforementioned plane.

8. A method of adjusting the quantity of a component measured by a photoacoustic monitoring device as claimed in claim 3, wherein
λ₁ is 532 nm
λ₂ is 558 nm; and
λ₀ is 545 nm.
